(19) United States
(12) Patent Application Publication
(10) Pub. No.: US 2019/0228842 A1
(43) Pub. Date: Jul. 25, 2019

(54) RECIPE FOR THE SYNTHESIS OF METASTABLE STRUCTURES USING TOPOLOGICALLY ASSEMBLED PRECURSORS

(71) Applicant: Virginia Commonwealth University, Richmond, VA (US)

(72) Inventors: Purusottam Jena, Richmond, VA (US); Hong Fang, Richmond, VA (US)

(21) Appl. No.: 16/192,965

(22) Filed: Nov. 16, 2018

Related U.S. Application Data
(60) Provisional application No. 62/587,559, filed on Nov. 17, 2017.

(51) Int. Cl. G16C 20/10; G16C 20/30; G16C 20/80; C01B 32/194

(52) U.S. Cl. CPC G16C 20/10; G16C 20/30; G16C 20/80; C01P 2006/40; C01B 2204/02; C01P 2004/20; C01B 2204/20; C01B 32/194

(57) ABSTRACT

Methods of planning and executing the synthesis of metastable materials are provided. Topologically assembled precursors having potential energy surfaces in which the volumes of potential wells of certain local minima are increased are created in silico. The precursor molecules are used to synthesize, e.g. two-dimensional metastable carbon materials such as penta-graphene comprised entirely of pentagons, O-graphene comprised of five- and eight-membered rings, and R-graphene comprised of four-, six- and eight-membered rings.

### STATEMENT OF FEDERALLY SPONSORED RESEARCH AND DEVELOPMENT

This invention was made with government support under grant number DE-FG02-96ER45579 awarded by the United States Department of Energy, Office of Basic Energy Sciences, Division of Materials Sciences and Engineering; and under grant number DE-AC02-05CH11231 awarded by the Office of Science of the United States Department of Energy. The United States government has certain rights in the invention.

### BACKGROUND OF THE INVENTION

### Field of the Invention

The invention generally relates to the synthesis of metastable structures. In particular, the invention provides methods of selecting precursors with a high probability of forming the metastable structures, and methods of synthesizing the metastable structures using the selected precursors in modeling as well as in experiments.

### Description of Related Art

Metastable structures provide us with a diversity of electronic and mechanical properties which, in many cases, are more desirable than those of the ground state [1-7].

However, limited experimental attempts have been made to synthesize metastable nanostructures. One example is the C₂₀ fullerene composed of only pentagons, which is metastable because of inherent strains [16]. The structure could not be spontaneously formed in carbon condensation or cluster annealing. However, it was synthesized [17] using a precursor that bears close resemblance to the cage structure, namely dodecahedrane (C₂₀H₂₀). Another example is the synthesis of graphdiyne (a metastable structure of two-dimensional carbon), which is synthesized on a metal substrate by using hexaethynylbenzene (C₁₈H₆) molecules that bears close resemblance to the building block of graphdiyne.

Theoretical approaches are yet to be developed to assist the experimental synthesis of metastable structures by deliberately creating potential energy surfaces (PES) in which the probability of forming the desired structures are enhanced. Current approaches mainly focus on structure prediction/search, such as simulated annealing [8-9], basin hopping [10-11], genetic algorithm methods [12-13], molecular packing [14], and data mining [15]. The methods merely explore the PES to identify structures with high realizability, measured by the volume (both the depth and width) of the potential wells of the local minima on the PES. Most metastable structures, especially the ones with high energy, are dismissed as hard-to-realize, because their potential wells are either too shallow or too narrow or both.

### SUMMARY OF THE INVENTION

Other features and advantages of the present invention will be set forth in the description of invention that follows, and in part will be apparent from the description or may be learned by practice of the invention. The invention will be realized and attained by the compositions and methods particularly pointed out in the written description and claims hereof.

Metastable structures of matter often possess properties superior to those of their ground state, e.g. diamond vs. graphite. Yet, in practice, many predicted metastable structures are dismissed as hard-to-realize as their corresponding local minima in the potential energy surface are either too shallow or too narrow or both. This disclosure provides methods that can enhance the realizability of targeted metastable structures deliberately and directly guide the experimental synthesis, as follows: First, a molecular precursor is identified according to its resemblance to the atomic, structural and local symmetry of the repeat unit of the targeted metastable structure; Second, different topological assemblies are achieved by confining the precursor units into a constrained superlattice with controlled overall orientation to induce connectivity between certain atomic nodes of the neighboring units; and Third, all the initial assemblies of the precursors are relaxed to their near energy critical point using density functional theory (DFT). The realizability of a structure is measured according to its frequency of occurrence in the relaxed Topologically Assembled Precursors (TAP). Thus, potential energy surfaces are created by design, in silico, in which the volumes of the potential wells of the metastable structures are increased, while suppressing the accessibility of structures of the ground state. The methods are applied to identify suitable precursors that can be used to synthesize high-energy metastable structures with exotic properties, such as 2D carbon allotropes, including penta-graphene comprised entirely of pentagons, O-graphene comprised of five- and eight-membered rings and R-graphene comprised of four-, six- and eight-membered rings. Crystalline solid materials prepared by these methods are also encompassed.

It is an object of this invention to provide a method of synthesizing a metastable crystalline material from a precursor comprising I) selecting the precursor by: i) identifying potential precursors, wherein the potential precursors are identified by—determining the number of atoms in a building block of the metastable crystalline material; —determining the types of bonds between the atoms in the building block; —selecting, from a molecular database, potential precursors having a) the same type of atoms as the builiding block, b) the same number of atoms as the builiding block, c) at least one bond of a type that is the same as at least one bond in the building block; —aligning the potential precursors; —selecting, as candidate precursors, potential precursors in which bonding between atoms of aligned neighboring potential precursors can occur; ii) for each selected candidate precursor, generating a set of different topologically aligned precursors (TAP); iii) geometrically and ionically relaxing each TAP to a closest critical point of the potential energy surfaces (PES); iv) calculating the frequency of occurrence of the metastable crystalline material within relaxed TAP; v) selecting at least one candidate precursor to be used as a precursor to synthesize the metastable crystalline material, wherein a frequency of occurrence of the at least one metastable crystalline material in the relaxed TAP of that candidate precursor is at least 2 times a frequency of occurrence of energetically similar metastable crystalline structures, and/or the ground-state of the metastable crystalline structure; and II) reacting the precursor to form the metastable crystalline material. In some aspects, the metastable crystalline material is a two dimensional (2D) or three dimensional (3D) metastable crystalline material, or an allotrope thereof. In some aspects, the metastable crystalline material comprises one or more of carbon, boron, nitrogen, phosphorus, silicon or a metal. In other aspects, the metastable crystalline material is a 2D carbon allotrope. In further aspects, the 2D carbon allotrope is penta-graphene, O-graphene or R-graphene. In some aspects, the step of geometrically and ionically relaxing is performed using density functional theory (DFT). In other aspects, the step of geometrically and ionically relaxing is performed while suppressing accessibility of the ground state and other isomers. In further aspects, each TAP is formed by contraining, within a superlattice, multiple copies of one candidate precursor. In additional aspects, each copy of the candidate precursor within the superlattice has the same fixed orientational configuration. In other aspects, the frequency of occurrence is at least twice the frequency of occurrence of energetically nearest neighbor structures and/or the ground-state structure. In further aspects, the frequency of occurrence is at least one order of magnitude higher than the frequency of occurrence of energetically nearest neighbor structures and/or the ground-state structure. In further aspects, the step of selecting comprises selecting potential precursors having at least one of: the same type of atoms as the building block of the metastable crystalline material, the same number of atoms as the building block of the metastable crystalline material, the same atomic orbitals as the building block of the metastable crystalline material, and the same size as the building block of the metastable crystalline material. In some aspects, the metastable crystalline material is pentagraphene and the precursor that is selected is 3,3-dimethyl-1-butene.

The invention also provides a method of selecting a precursor for synthesis of a metastable crystalline structure, comprising i) identifying potential precursors, wherein the potential precursors are identified by—determining the number of atoms in a building block of the metastable crystalline material; —determining the types of bonds between the atoms in the building block; —selecting, from a molecular database, potential precursors having a) the same type of atoms as the builiding block, b) the same number of atoms as the builiding block, c) at least one bond of a type that is the same as at least one bond in the building block; —aligning the potential precursors; —selecting, as candidate precursors, potential precursors in which bonding between atoms of aligned neighboring potential precursors can occur; ii) for each selected candidate precursor, generating a set of different topologically aligned precursors (TAP); iii) geometrically and ionically relaxing each TAP to a closest critical point of the potential energy surfaces (PES); iv) calculating the frequency of occurrence of the metastable crystalline material within relaxed TAP; v) selecting at least one candidate precursor to be used as a precursor to synthesize the metastable crystalline material, wherein a frequency of occurrence of the at least one metastable crystalline material in the relaxed TAP of that candidate precursor is at least 2 times a frequency of occurrence of energetically similar metastable crystalline structures, and/or the ground-state of the metastable crystalline structure.

The invention further encompasses a compound having a metastable crystalline structure made from a precursor that is selected as described herein. In some aspects, the compound is selected from the group consisting of penta-graphene, O-graphene and R-graphene.

### BRIEF DESCRIPTION OF THE DRAWINGS

FIG. 1A-D. Types of carbon in A, penta-graphene, B, R-graphene, C, O-graphene and D, graphene. Different types of carbon according its local symmetry are categorized into sp¹, sp², sp³, and sp²d¹ configurations. Carbon atoms are denoted by C(1), C(2), C(3), C(4) and C(E), where C(1) is the one atom eclipsed by the C(E) atom when looking down the three-fold rotational axis of the precursor molecule. The arrows show the connections needed between neighboring atomic nodes to form the targeted allotropes. Bond breaking (in double dashes) is needed to form R-graphene, O-graphene and graphene which are comprised solely of sp² carbon. Rotation of the units during the relaxation is involved in forming graphene.

FIG. 2A-D. A, candidates for precursor molecules to realize penta-graphene, R-graphene and O-graphene. Also shown are the lateral views (y) when the precursors are aligned. Carbon atoms are denoted by C(1), C(2), C(3), C(4) and C(E), where C(1) is eclipsed by C(E) or staggered by C(S) when looking down the three-fold axis (C₃) of the molecule. 3,3-dimethyl-1-butene has three rotational degrees of freedom and can be viewed as a dipole vector plus a branch in symmetry, as shown by the schematics; B, the view of penta-graphene down the z and y axes; C, different rotational potentials of 2,2-dimethylbutane and 3,3-dimethyl-1-butene looking down C₃, where the molecules adopt a staggered configuration and an eclipsed configuration in their ground states, respectively; D, the computational model adopted in the approach.

FIG. 3. The relative probability of different structures vs. their energy above the ground state. Peaks of different structures are identified with their potential wells shown in FIG. 4. Penta-graphene, R-graphene and O-graphene all have high realizabilities. The realizability of graphene is suppressed as indicated by its tiny peak. Peaks in the area beyond 1.2 eV/atom correspond to the states as high peaks (large darkened areas) in FIG. 4, which are not local minima.

FIG. 4A-D. Calculated PES using the TAP. β, α and γ are the rotational angles about the x, z and D axes, see FIG. 2D, respectively. The shading scheme corresponds to the relative energy of various structures compared to penta-graphene. A, β=5 degrees and 10 degrees; B, β=−5 degrees and 0 degrees; C, β=15 degrees and 30 degrees; D, β=45 degrees, 60 degrees and 75 degrees.

FIG. 5. Work-flow chart of our approach in sampling structures using TAP with different orientational configurations.

FIG. 6A-C. Carbon-carbon connections need to be made to form A, penta-graphene, B, Q-graphene and C, E₂-graphene.

FIG. 7. Charge states and dipole moment (arrow) of C₆H₁₀Br₂. Br is in black, C in grey and H in white.

FIG. 8A and B. A, optimized structures of the precursor molecules for the Grignard reaction with the numbers showing the charge states of different carbon atoms. B, calculated reaction pathway of the Grignard reaction with configurations of the reactant, the transition state and the product. The arrows show the vibration of the single imaginary mode of the transition state. Bris in black, Mg is crosshatched, C is in grey and H is in white. The free energy barrier ΔG of the reaction is calculated as ΔG=G^{TS}−G^{react}=1.78 eV, where G^{TS} and G^{react} are the free energies of the transition state and the reactant, respectively.

FIG. 9. Flow chart of steps of the method.

### DETAILED DESCRIPTION

The present disclosure describes methods to identify suitable precursors and their topological assemblies for the synthesis of metastable structures. Generally, the metastable structures are crystalline solids that comprise (are made up of) of repeating subunits (e.g. identical repeating subunits or building blocks). In some aspects, the metastable structures occur as or are in the form of flat (planar) two-dimensional “sheets” of interconnected, covalently or ionically bonded subunits. However, the methods described herein are also applicable to the synthesis of three-dimensional metastable structures.

In contrast to prior art structure-prediction techniques, which use individual atoms to develop a relatively comprehensive configuration space which can be explored to locate global minima, the present methods are based on the selection of one or more suitable precursors, each of which is a cluster of atoms with established bonding symmetry. This strategy permits the creation of potential energy surfaces (PES) based on the potential precursors forming different topological assemblies in silico, and to find those whose formations involve less structural reconstruction and are thus more likely to occur during a synthesis, since all chemical changes have a certain associated energy barrier to overcome. The present method focuses on the realization of metastable structures with high energy, while the traditional single atom approach is focused on finding the ground-state structure with the global minimum energy. These present methods thus represent a streamlined process as compared to the current more arduous single atom approach which often leads to many non-productive results.

### Definitions

“Crystal” or “crystalline solid” is a solid material whose constituent atoms are arranged in a periodic arrangement, forming a highly ordered microscopic crystal lattice. The individual subunits are unit cells containing one or more atoms in a specific spatial arrangement. The unit cells are arranged as a sheet for two-dimensional crystals or as stacked “boxes” for three-dimensional crystals.

“Metastable” refers to a stable state of a dynamical system whose energy is higher than the global minimum of the system.

“In silico” refers to experiments or research conducted or produced by means of computer modeling or computer simulation.

“Potential energy surface” (PES) refers to an energy surface, where energies of structures with different arrangements of atoms are shown.

“Atomic node” refers to the atom available to form bonding with others.

“Isomer” and “Allotrope” are alternative names for the “metastable structures” described herein.

“Topologically assembled precursors” (TAP) refers to an assembly (plurality) of replicated, aligned precursor molecules in a superlattice. Each precursor molecule in a given TAP has the same orientational configuration. Generally, a plurality of TAP is created and in each TAP, the orientational configuration of the precursor molecules differs from that of the other TAP.

Types of Metastable Structures that can be Synthesized

A plethora of metastable structures may be synthesized using the methods described herein. Those of skill in the art will recognize that the methods described herein may be used to synthesize any type of solid crystalline compound. Examples include but are not limited to those made of carbon, boron, nitrogen, phosphorus, silicon, various metal elements, etc., or those made up of a mixture of different types of atoms (e.g. alloys), as well as allotropes thereof. The solid crystalline compounds can have 2D or 3D structures. In the description below and in the examples, the metastable structures are 2D carbon allotropes. However, examples of other compounds that can be synthesized using the methods described herein include but are not limited to: three-dimensional crystal solids such as 3D metallic carbon structures and 3D semiconducting carbon structures.

### Identifying the Subunit of a Metastable Structure

Metastable structures (e.g. compounds) that are targeted for production contain identifiable repeating subunits or building blocks (motifs, unit cells, repeat units, etc.). Each subunit is a contiguous group or local pattern of atoms that is repeated (occurs or recurs periodically) in all dimensions within the structure. Such unit cells are the smallest part of a crystal that has the two- or three-dimensional pattern of the whole lattice. This is the smallest repeating unit in the lattice. For example, in a 2D carbon structure, a subunit is a group of contiguous C atoms which, when multiple copies of the subunit are aligned as a two-dimensional sheet (in an x-y plane), form the pattern that is characteristic of the metastable structure. For a 3D carbon structure, the subunits are aligned side by side in the x-y direction and also stacked in the z direction.

A step of identifying the subunit of a metastable structure may be performed by a skilled practitioner “by eye” e.g. using molecular modeling simulations and recognizing the repeated patterns. Usually, computer software programs are used to interrogate parameters of known crystals to identify the subunits. For example, the Cambridge Structural Database (CSD) is a highly curated and comprehensive resource for small-molecule organic and metal-organic crystal 3D structures, and the Crystal Structure Prototype Database (CSPD), Computational 2D Materials Database (C2DB), and Crystallography Open Database (COD) are databases with search functions to identify the unit cells of crystalline structures.

### Selecting Possible (Potential) Precursors

At least one potential molecular precursor is identified (selected) as a possible candidate to be used as the basis of forming one or more multiple metastable structure(s). Potential precursors are identified according to their resemblance to the atomic, structural and local symmetry of the subunits of the targeted metastable structure. In other words, each potential precursor is generally selected according to its resemblance to the repeating subunits of the metastable structure. For example, a suitable potential precursor would typically contain the same types of atoms (e.g. C) and the same number of atoms as does a repeating subunit of the metastable structure, and the atoms of a potential precursor would be arranged in the same contiguous bonding pattern as those of the subunit. Typically, at least one of the atoms of the potential precursor has the same type of atomic bonding symmetry as does at least one of the atoms of the subunit, e.g. if the subunit, when present in the targeted metastable structure, has at least one sp³ C atom, then a potential precursor also has at least one sp³C atom. Typically, a suitable precursor also has a size that is comparable to that of the repeating subunit of the metastable structure, e.g. the dimensions of the precursor are the same within about +/−1.0 Å or less, e.g. within about +/−0.5 Å. The size similarity generally follows as a consequence of the desired number of atoms and the bonding pattern.

A selected precursor compound typically differs from the repeating subunit of the metastable structure in that in a natural, or a chemically allowable or a preferred stable state, the selected compound comprises atoms which are not present in the metastable structure of interest. In other words, a selected precursor compound typically differs from the repeating subunits of the metastable compound in that it has a similar parent chain but different substituents. For example, in some aspects, a known compound is selected which comprises H and halogens, etc., or other atoms or functional groups linked to a parent chain (e.g. a skeleton of carbon atoms) and it is only the atoms of the parent chain that represent the subunit and will be present in the metastable structure that is synthesized. Thus, a possible precursor as defined herein comprises only the atoms of the parent chain. The substituents(e.g. leaving groups) have been “removed” from the compound in silico to create a virtual construct and it is the virtual construct that serves as the potential precursor in order to test (model), in silico, the feasibility of using the compound to synthesize the targeted metastable structure. However, it is also possible to remove the extraneous atoms from the precursor in reality (e.g. experimentally) e.g. by using one or more catalysis-aided chemical reactions under certain reaction conditions. In a laboratory setting, experimental removal of these leaving groups (functional groups) may occur all at once, but more often removal occurs in a step-wise fashion. That is, during a synthetic reaction to synthesize the metastable compound using the precursor compound, at each step of the reaction, one or more of the atoms or atomic groups of the precursor is removed and is thus free and available to form a bond with one or more atoms of the neighboring precursor compounds. The original bonds between the parent chain and the leaving groups of the precursor are replaced by bonds to adjacent parent chains. Those of skill in the art will recognize the feasibility of the chemical transformation, e.g. during a gas phase synthesis in which the precursor compound is present at a controlled vapor density.

Possible precursors are selected based on interrogations of databases. Examples of databases that are searched include but are not limited to: ChemDB, the Cambridge Structural Database, the GDB-10 and GDB-13 databases at the University of Bern, and others known in the art. In these databases, it is possible to search using the atomic structure of the subunit of the structure that is to be synthesized. The search may be set at various levels of stringency to match the type and bonding patterns of the relevant atoms, in order to identity possible real-world compounds that could function as precursors.

### Selecting Candidate Precursors

In addition, as another criterion, e.g. for a 2D material, when multiple copies of a potential precursor are aligned side-by-side, e.g. in a “sheet”, the atoms at the edges of neighboring, adjacent potential precursors should be capable of being sterically positioned and should have suitable electronic properties so that one or more bonds like those of the metastable structure can be formed between adjacent potential precursors. In other words, the adjacent atoms should be theoretically capable of forming new chemical bonds identical to those that occur between the repeating subunits of the metastable structure; orbital overlap/hybridization should generate or reproduce the bonds that are present between the repeating subunits of the metastable structure. The current method can identify the particular atomic connection(s) needed in order to form (or at least increase the probability of forming) a targeted metastable structure from the precursor molecules.

For 2D materials, this property of a potential precursor unit is tested in silico by aligning the potential precursors into a sheet and determining whether or not it is possible to “fit” them to the desired bonding pattern, e.g. a pattern that closely resembles or is that of the metastable structure. If, for example, the dihedral angles inside a potential precursor molecule do not permit a key connection between neighboring units (for example, if the angle prevents atoms in adjacent precursor molecules from bonding with each other because the distance between them is too large, i.e. they are not within bonding distance), then the candidate is rejected (discarded), even if other criteria (such as number of atoms, etc.) have been met. However, if proper alignment is possible, then the potential precursor is selected as a candidate precursor for further testing. For example, for carbon-carbon bonding, a distance of from about 1.3 to about 1.6 Å between neighboring potential precursors should be possible, such as about 1.1, 1.2, 1.3, 1.4, 1.5, 1.6, 1.7, 1.8, 1.9 or 2.0 Å, and usually about 1.3, 1.4, 1.5, or 1.6 Å.

For 3D structures, the procedure and requirements (e.g. bond distance requirements) are generally the same but the alignment requirements must be met in three-dimensions. The selected precursor molecules are oriented in the 3D superlattice with different stacking sequences.

Likewise, if non-carbon atoms are present in the metastable structure, and thus also in the potential precursors, those of skill in the art will recognize that potential precursors will be oriented and evaluated using the same principles.

### Generating Topologically Assembled Precursors (TAP)

Once one or more candidate precursors are identified, in order to assess the effect of different stereochemical orientations on the likelihood (probability) of success in synthesizing the metastable structure from a given precursor, different assemblies of the precursor molecules with different overall orientations are generated within a superlattice. In particular, for each candidate, three rotational axes are selected to exhaust the degrees of freedom of the precursor molecule as a rigid body: one in the x direction, one in the z direction and a third, D, in the direction of the intrinsic dipole moment of the molecule. Each candidate precursor is then rotated by one set of randomly-generated angles (e.g. β, α and γ) about the three axes x, z and D, respectively, to form one fixed orientational configuration of the candidate precursor.

Within a superlattice, for a 2D material, the configured unit (a candidate precursor with one defined orientational configuration) is duplicated (replicated) in the x-y plane along (1,0), (1,1), (0,1), (−1,1), (−1,0), (−1,−1), (0,−1) and (1,−1) directions, resulting in a group of topologically assembled precursors (a “TAP”) within the superlattice, all of which have the same orientational configuration. For a 3D material, besides such alignment of the configured units along the x and y directions, they are also stacked along the z direction. Multiple sets of different randomly generated angles are typically used for each candidate precursor so that different TAP are formed e.g. from about 500 to 5,000 TAP are formed for each candidate precursor. For example, about 500, 600, 700, 800, 900, 1000, 1500, 2000, 2500, 3000, 3500, 4000, 4500 or 5000 TAP may be formed for each candidate precursor. With each TAP, all the precursor molecules are aligned and have the same defined orientational configuration, and the defined orientational configuration differs for each TAP.

Thus, multiple copies of a single candidate precursor are aligned and constrained (in silico) in the Cartesian coordinate system of a superlattice, and within the superlattice, each copy of the candidate precursor has the same stereochemical orientation about all possible bonds. The individual candidate precursors are aligned e.g. as a 2D sheet, or as a 3D structure, depending on the metastable structure that is to be synthesized. Generally, from about 9 to 125 copies of a candidate precursor are aligned within the superlattice, e.g. about 9, 27, 64, 125, or more copies are aligned in an x-y plane for 2D structures and in the x, y and z directions, for 3D structures. In some aspects, such as for 2D structures, at least 9 independent aligned precursor molecules are needed for the computations and simulations so that all possible connections of a central precursor to its neighboring precursors is shown. Otherwise, the calculation is flawed, because a periodic boundary condition is applied, and a limited number of neighbors does not permit identification of all the possible connections. For example, at least 9 copies may be used for a 2D material and at least 27 for the case of 3D, where one has at least one precursor molecule in the center to have all the possible connections with its neighbors. 64 and 125 and generally are for larger supercells.

It is noted that if two or more candidate precursors are identified, then 2 or more sets of 500 to 5000 TAP will be generated, one set of 500 to 5000 TAP for each candidate precursor.

### Relaxation of TAP

Each TAP is then fully relaxed (both geometrically and ionically) to its nearest critical point of energy using density functional theory (DFT) calculations. Those of skill in the art are familiar with DFT simulations/computations, as described, for example, in U.S. patent application Ser. Nos. 10/109,760 and 10/096,489, which represent the more than 450 patents related to DFT filed per year since 2015. The complete contents of each of these references, and references cited therein, are hereby incorporated by reference in entirety. Each relaxation will provide a critical point on the potential energy surface (PES) and, after the relaxation of all the TAP, the whole PES is mapped out.

### Selecting Precursors

The probability of forming (likelihood of formation of) a metastable structure using a candidate precursor is measured according to its frequency of occurrence in the set of relaxed TAP. If the frequency of occurrence is high, then the candidate precursor is deemed to be a suitable precursor for synthesizing the targeted metastable structure.

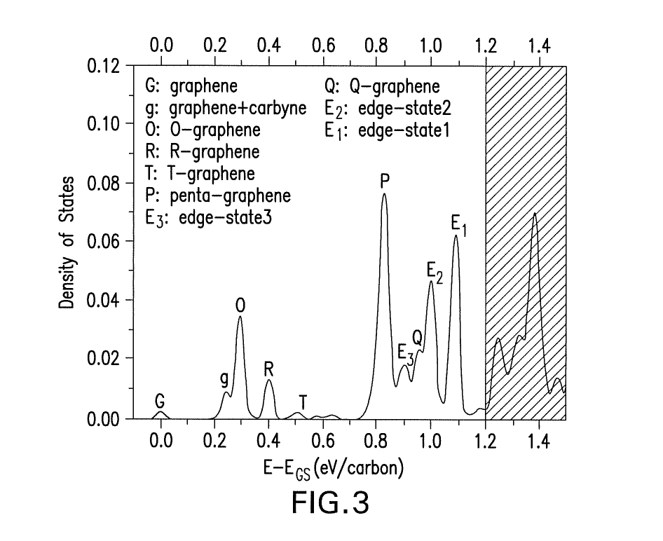

The “frequency of occurrence” is measured e.g. by counting how many TAP out of the total will result in a particular metastable structure after relaxation. It is equivalent to the volume of each “potential well” occupied by that particular metastable structure in the PES. By comparing the frequency of occurrence for different metastable structures, one can obtain which metastable structure(s) is (or are) most likely to form using the selected precursor; and which TAP can result in a particular metastable structure, which can directly guide experimental synthesis. In general, a “high” frequency of a metastable structure is defined as several times to more than an order of magnitude higher than the frequency of its energetically neighbored structures and the ground-state structure (e.g. see FIG. 3). For example, the frequency of occurrence may be 2-100, 200, 300, 400, 500, 600, 700, 800 or 900× times higher, or even one or more orders of magnitude higher, than i) the frequency of structures that are the nearest in energy and/or ii) the ground state structure. In addition, the analysis does not need to be “all or nothing” in that metastable structures can be ranked from high to low. For example, if a metastable structure exhibits a frequency of occurrence of less than about 1%, then this structure is considered as not likely to be formed by the selected precursor and the corresponding TAP. If a metastable structure exhibits a high (or at least higher) frequency of occurrence compared to those of other possible metastable compounds, then it is known that the selected precursor can be used to synthesize this particular metastable structure. The corresponding TAP offer direct guidance to the experimentalists on how to align the precursor molecules so that the metastable structure is synthesized.

Other factors can also impact the selection of a precursor for experimental synthesis and make a candidate more desirable or realistic, even if it is not ranked highest in a group in terms of realizing the targeted metastable structure. Such factors include availability of the precursor, the cost of the precursor, the ease of manipulation of the precursor, toxicity, ease of adapting the precursor to a real-world synthesis (e.g. availability of suitable reaction conditions), etc. Thus, when choosing among multiple possible candidate precursors, while the frequency of the representation or occurrence of the metastable structure in relaxed TAP is likely most important, other factors can impact the selection. Further, adjustments to reaction conditions may compensate for lower ranking candidates, e.g. an increase in the concentration of a reactant, an increase in reaction temperature, an increase in reaction time, etc. may be used to bias the synthetic reaction toward the desired outcome, i.e. successful synthesis of the metastable structure of interest.

In some aspects, it is possible that only one candidate precursor is identified. In this case, it is necessary to make a judgement call on whether or not the frequency of occurrence of the metastable compound is sufficient to warrant the use of the compound as a viable precursor. In general, the selected precursor should result in a metastable structure whose frequency of occurrence is several times to more than an order of magnitude higher than the frequency of its energetically neighbored structures and the ground-state structure (e.g. see FIG. 3).

As shown in FIG. 3, penta-graphene, O-graphene and R-graphene all have high probability to be formed using 3,3-dimethyl-1-butene as the candidate precursor, while the probability of realizing graphene and many other possible isomers are suppressed using this precursor.

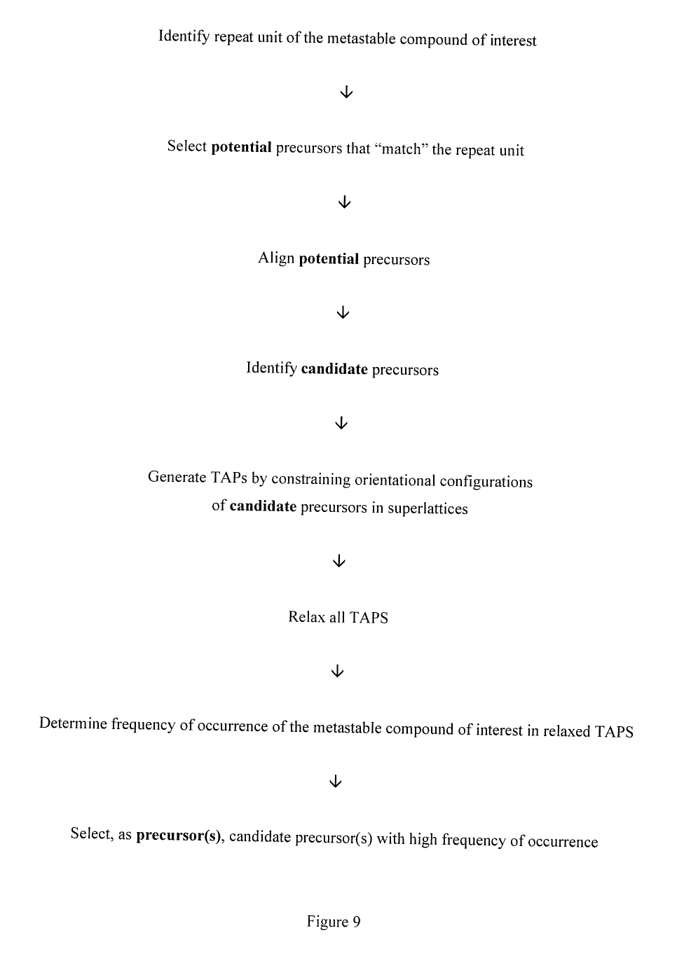

It is noted that some precursors may be useful for the synthesis of more than one metastable compound, and/or more than one metastable structures may be formed when a precursor is reacted. By controlling the alignment of the precursor molecules, namely using a particular set of TAP, a particular metastable structure can be formed. A flow chart outlining the broad steps of the method is shown in FIG. 9.

### Computer Programs

Generally, computer programs (e.g. software) are developed to cause a computer to perform the steps of the methods described herein. Example 4 provides a list of steps that would be included in a typical program. Input can include, e.g., the atomic structure of a metastable crystalline structure and rules to identify repeat units therein; instructions to access a database and select suitable potential precursors, e.g. by matching the type and number of atoms in the repeat unit, the types of bonds, etc. as described herein; and instructions to remove the leaving groups and align the parent chains of the potential precursors to determine whether or not distances between the atoms of neighboring parent chains are appropriate to form bonds between them. If not, the candidate is rejected; but if distances are suitable, then further instructions cause the program to align the parent chains in a superlattice to form a plurality of TAP for each candidate precursor; instructions to relax each TAP to form a PES; and instructions to identify occurrences of (determine the frequency of occurrence of) the metastable crystalline structure within the PES. Rules containing cut-off or threshold values for eliminating or accepting candidate precursors as viable precursors based on the frequency of occurrence are generally also provided in the program. Typically, one program is developed to perform all steps of the method. Alternatively, more than one program may be developed, with one performing some of the steps (e.g. identifying unit cells of the metastable crystalline material) and integrated with one or more other programs so as to output information that is input into the next program, which in turn performs the next steps of the method, etc., i.e. the programs are used successively.

### Experimental Synthesis of Metastable Compounds

Once a suitable precursor has been identified, it can be used to synthesize the targeted metastable structure. Those of skill in the art will recognize that the conditions required to synthesize a targeted metastable structure using a precursor identified herein are readily available and in current use. For example, the targeting of one or more non-carbon atoms in a precursor having a skeleton comprised of carbon for removal, and hence, availability to bond to another precursor in a stereochemically correct or desired manner, is well-known. Examples include e.g. the use of halogenated compounds from which halogen atoms are preferentially added and then removed from selected carbon sites. Other reactions that can induce the connection between certain atomic sites include but not limited to the Grignard reaction; enolate alkylation; aldol concentration reactions; Claisen condensation; Michael reaction; pinocol coupling reactions; various sn1 reactions, etc.

In addition, reaction conditions that are more stringent or harsh than those used with reactions that are readily energetically favorable may be needed, and many useful techniques are available. For example gas phase syntheses are known as are syntheses that use, for example, heat and/or microwaves to drive a reaction. External fields such as electric field and/or laser pulses can be used to control the orientations of the precursor molecules during the reaction. The average distance between the aligned precursor molecules can be controlled by the vapor density in gas phase syntheses. In addition, particular catalysts can be used to facilitate the needed reaction, e.g. dehydrogenation of the precursor compound, as well as to help align the precursor molecules to form the desired TAP.

Compounds that can be Synthesized

Metastable allotropes (which are sometimes exotic metastable allotropes that have not been previously synthesized) that can be synthesized by the methods described herein include but are not limited to:

Carbon allotropes such as penta-graphene (comprised entirely of pentagons), O-graphene (comprised of five- and eight-membered rings) and R-graphene (comprised of four-, six- and eight-membered rings);
Silicon allotropes such as the BC8 allotrope;
Germanium allotropes e.g. germanium ST12, m-allo-Ge and 4H—Ge;
Phosphorous allotropes, e.g. white phosphorus or yellow tetraphosphorus (P4), red phosphorous;
Boron allotropes such as α-rhombohedral boron, β-rhombohedral boron, γ-orthorhombic boron, α-tetragonal boron, β-tetragonal boron;
Nitrogen allotropes such as N6 crystals;
Selenium allotropes having 6, 7 or 8 membered rings;
and others that will occur to those of skill in the art.

It is to be understood that this invention is not limited to particular embodiments described herein above and below, and as such may, of course, vary. It is also to be understood that the terminology used herein is for the purpose of describing particular embodiments only, and is not intended to be limiting.

Where a range of values is provided, it is understood that each intervening value between the upper and lower limit of that range (to a tenth of the unit of the lower limit) is included in the range and encompassed within the invention, unless the context or description clearly dictates otherwise. In addition, smaller ranges between any two values in the range are encompassed, unless the context or description clearly indicates otherwise.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### EXAMPLES

Example 1

A process to enhance the realizability of targeted metastable structures, especially for 2D carbon allotropes, has been developed. The volumes of configuration space occupied by local minima in the PES, that correspond to the desired structures, are increased by using the topologically assembled precursors (TAP) and denying or limiting the accessibility of the ground state and other isomers. This can create PES in which structures whose formations involve less structural reconstruction in the precursor are more likely to occur, since any change has certain energy barrier to overcome. Due to different possible connections between the neighboring precursor molecules more than one metastable structure may form, starting from the same precursor. Aligning all the molecules into given orientation, defined as TAP, can trigger connections between certain atomic nodes upon relaxation, resulting in the targeted structure(s).

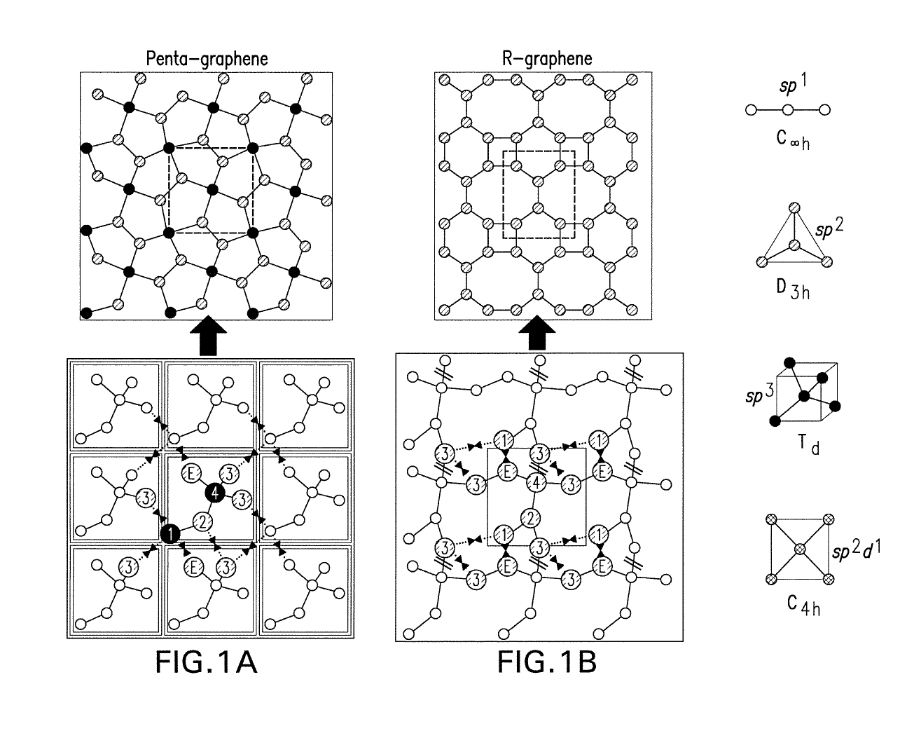

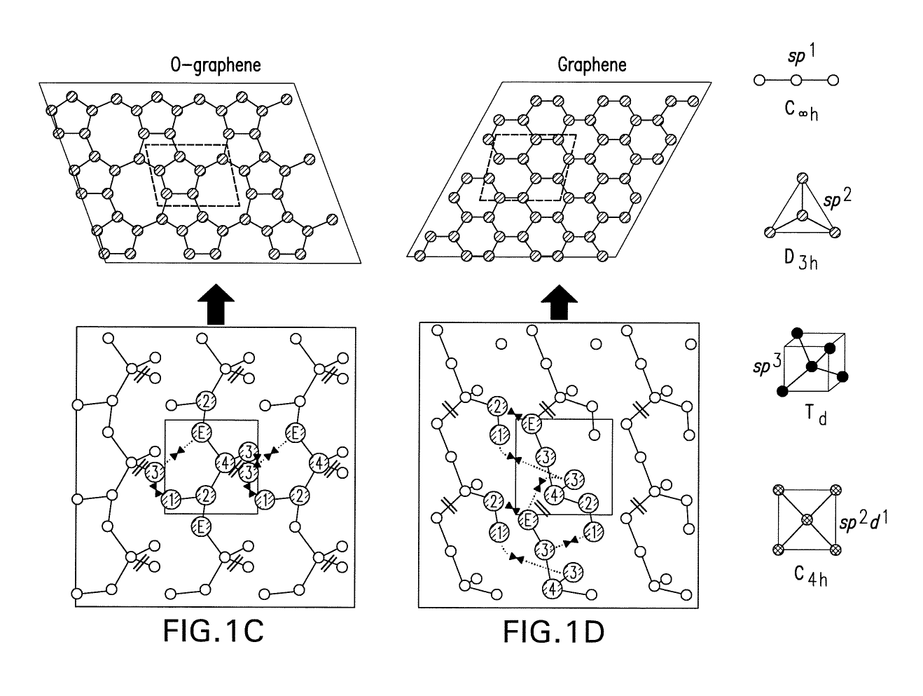

The method is demonstrated using penta-graphene comprised entirely of pentagons; O-graphene comprised of five- and eight-membered rings; and R-graphene comprised of four-, six- and eight-membered rings (see FIG. 1). All the allotropes have significantly higher energy than that of graphene (the ground state) and would be considered as hard-to-realize. Both penta-graphene and O-graphene, although being predicted to have novel electronic properties [19-20], have not yet been synthesized. The nanoribbons of R-graphene have only been synthesized by using biphenylene as a precursor and functionalizing carbon on selected sites [21]. Penta-graphene is especially interesting due to its exotic electronic and mechanical properties, such as a large bandgap, ultrahigh ideal strength, negative Poisson's ratio and chirality independent electronic structure [19]. However, penta-graphene is much higher in energy (0.82 eV/atom) than graphene and has been considered as experimentally unachievable [22]. FIG. 1 illustrates that each metastable structure mentioned above has a building block containing six carbon atoms. According to its local symmetry, each atom in the building block can be categorized as forming sp¹, sp² or sp³ bonds. Among the six atoms in the building block of penta-graphene, each sp² carbon is directly connected to one of its kind and surrounded by four sp³ carbon. All the carbon atoms of R-graphene and O-graphene are sp².

The following criteria are applied to the molecular database to find the right precursor: First, it should have six carbon atoms, of which at least one should be sp³.

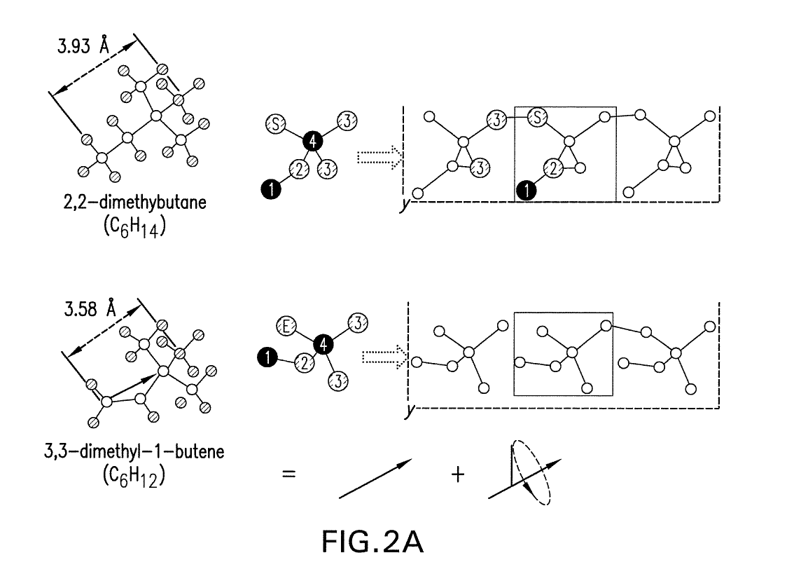

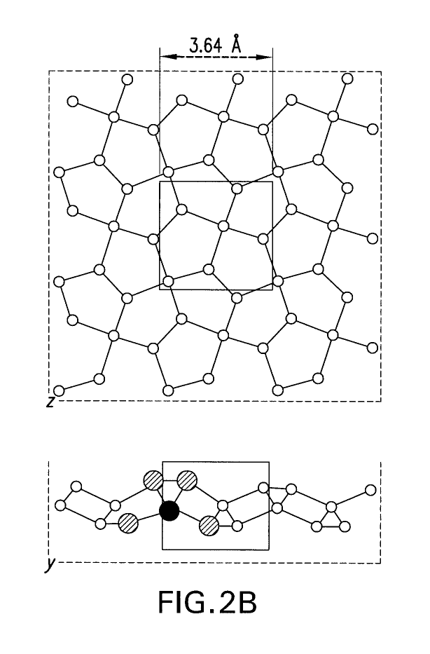

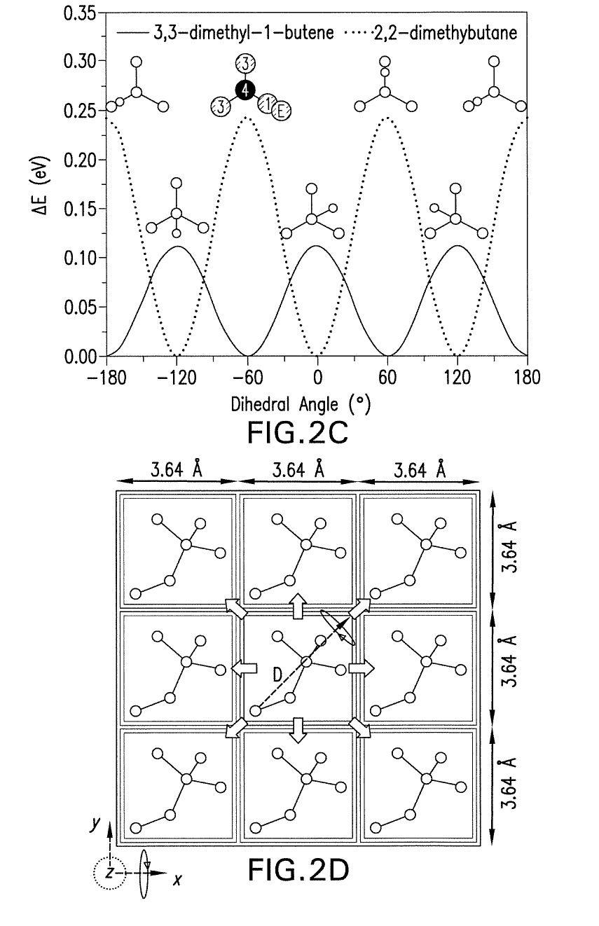

Second, its size should be comparable to those of the building blocks of the allotropes, which are in the range of [3.5, 4.5] Å. The candidates that fit these criteria are 2,2-dimethylbutane (C₆H₁₄) and 3,3-dimethyl-1-butene (C₆H₁₂) (FIG. 2A). Each molecule has one sp³ carbon and their sizes are around 4.0 Å. When the molecules are aligned into a superlattice, as shown in FIG. 2B, the C(1)-C(2)-C(4)-C(S) dihedral angle of 2,2-dimethylbutane is too large, preventing connections between the C(1) and C(3) atoms of the neighbors to make C(1) the second sp³ carbon required by penta-graphene. In contrast, 3,3-dimethyl-1-butene adopts an eclipsed configuration for its ground state (see FIG. 2C), resulting in an appropriate C(1)-C(2)-C(4)-C(E) dihedral angle. The molecule has two unsaturated hydrocarbon atoms C(1) and C(2) forming a double bond, which can enhance the realizabilities of R-graphene and O-graphene that are comprised entirely of sp² carbon, as will be discussed later.

With 3,3-dimethyl-1-butene as the selected precursor, we established a computational model, as shown in FIG. 2D, to build different topological assemblies of the precursor units. First, one 3,3-dimethyl-1-butene molecule is rotated by randomly-generated angles β, α and γ about three chosen axes, respectively, which are the x and z in the Cartesian system and D along the intrinsic dipole moment of the molecule. As shown in FIG. 1A, the molecule can be represented by an oriented dipole vector with a branch that destroys the infinite-rotational symmetry along the vector. β and α determine the orientation of the dipole vector and γ exhausts the third rotational degree of freedom. Next, the rotated molecule is duplicated in the x-y plane along (1,0), (1,1), (0,1), (−1,1), (−1,0), (−1,−1), (0,−1) and (1,−1) directions. The TAP is then fully relaxed (both geometrically and ionically) to the closest critical point of the PES using DFT. The above procedure is repeated to sample structures using TAP with a total of 1000 different orientational configurations. The realizability of each structure is measured by its frequency of occurrence, as shown in FIG. 3. penta-graphene, O-graphene and R-graphene all have high realizabilities, while the realizability of graphene and many other possible isomers are suppressed.

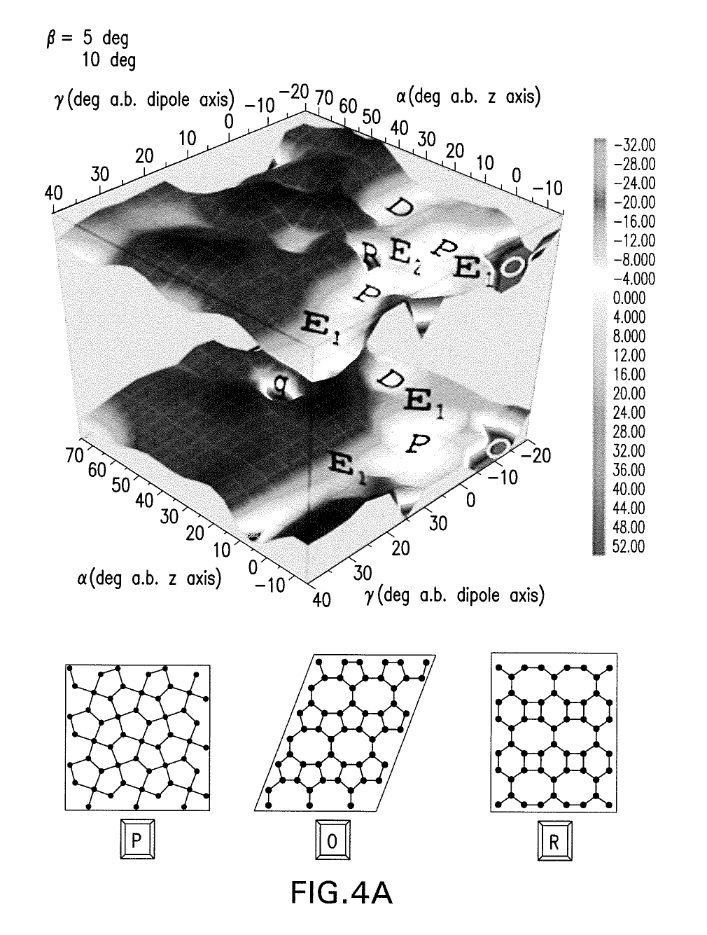

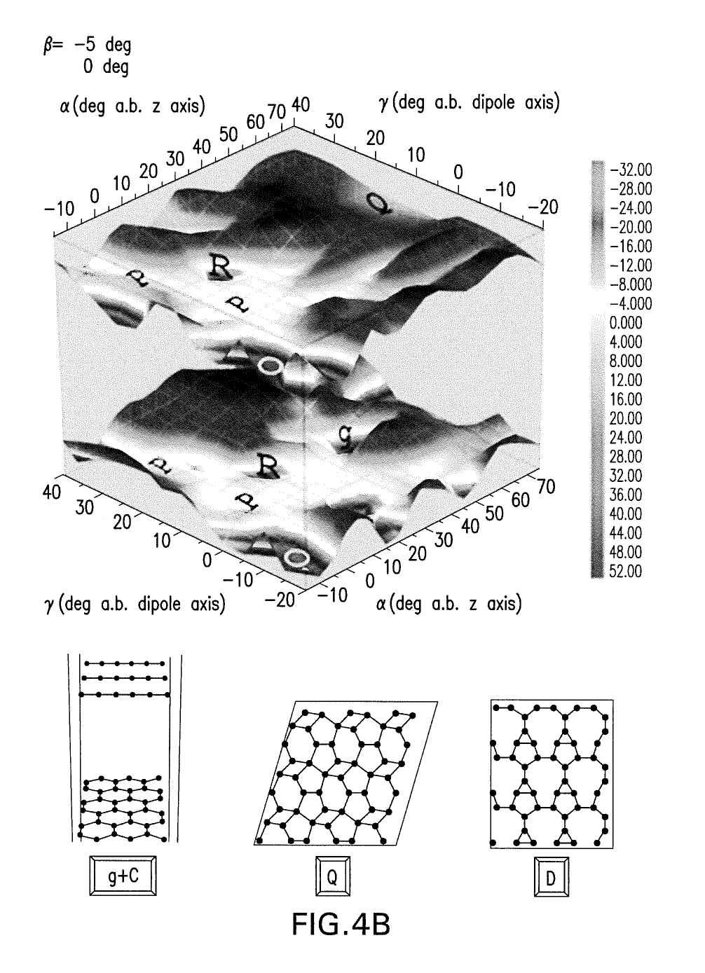

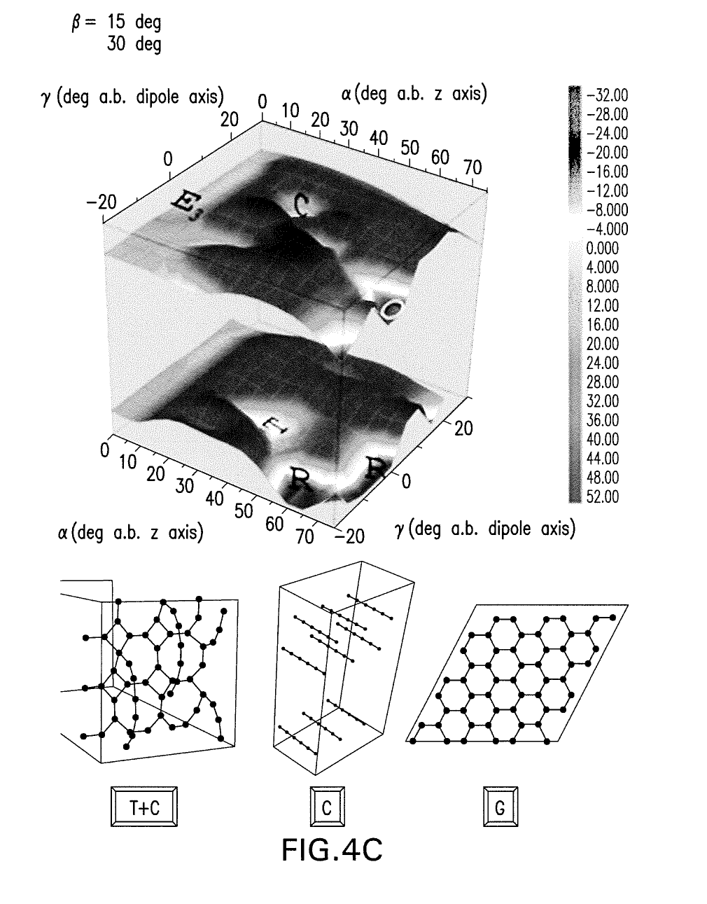

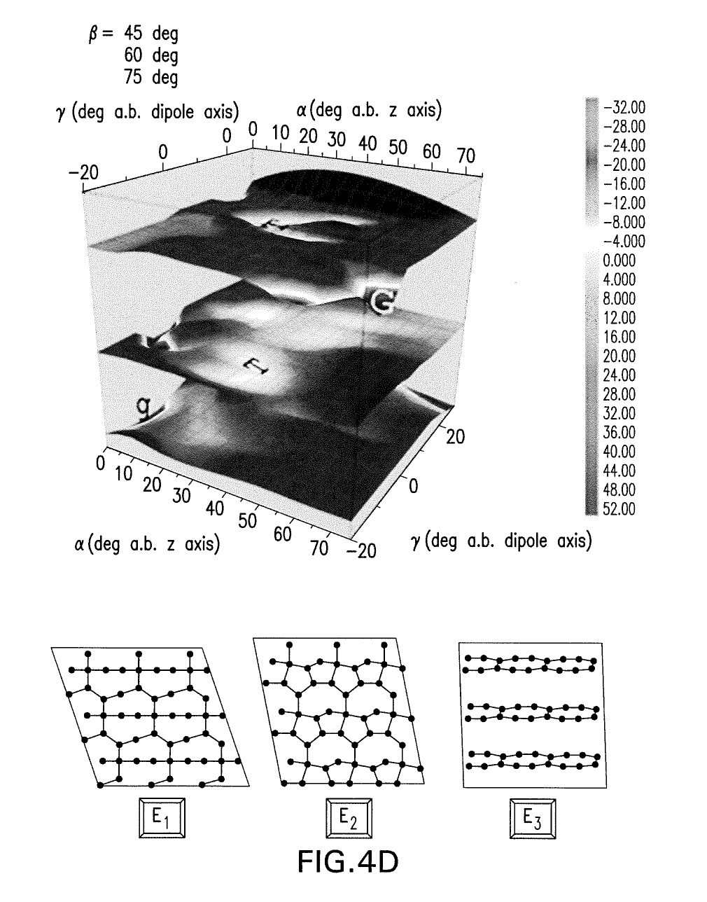

In FIG. 4, large areas of penta-graphene (P) are identified at β=−5°, 0°, 5° and 10°. In the range of [β=−5°, α=−10°˜15°, γ=0°˜40° ], [β=0°, α=−10°˜15°, γ=−10°˜30°] and [β=5°, α=−10°˜10°, γ=−5°˜20°], penta-graphene appears in extended valleys surrounded by E₁ and E₂ states whose energies are 0.30 and 0.20 eV/atom higher, respectively. The depressions in the vicinity of each valley correspond to R-graphene and O-graphene whose energies are 0.42 and 0.52 eV/atom lower than that of penta-graphene, respectively. In addition to the small depressions near penta-graphene, O-graphene and R-graphene mainly appear in isolated basins at relatively large β (=15° and 30°), when the D axis of the precursor is moderately out of the x-y plane.

Structures corresponding to other peaks in FIG. 3 are well separated from penta-graphene, R-graphene and O-graphene in the PES, highlighting the power of this approach. At β=−5°, there is a basin of Q-graphene comprised of four-, five- and six-membered rings whose energy is 0.14 eV/atom higher than that of penta-graphene. Large valleys of T-graphene comprised of four- and eight-membered rings [28] with some carbyne segments are found at large P. They constitute a set of small peaks in FIG. 3. At β=15°, carbynes that are linear chains of sp² carbon appear in an area surrounded by high peaks. Graphene only appears at the edge of the landscape with β=45°, which is consistent with its tiny peak in FIG. 3. The peaks beyond 1.2 eV/atom correspond to high energetic non-stable states (darkened areas) on the energy map.

Once the targeted metastable structures are established on the created PES, the precursor and the TAP needed to realize the structures are found, which can directly guide the experimental synthesis. There are several experimental tools to manipulate the orientation of the precursor molecules into the required topological assembly. One is to use an electric field (EF) to align the molecular dipoles. The magnitude of the EF is estimated as the value needed to overcome the thermal energy at a given temperature at which, in a typical experimental setting, the molecules with controlled vapor density are undergoing dehydrogenation on a catalytic surface. For instance, if 700 K is enough for dehydrogenation, then the magnitude is E=k_{B}T_{700K}/D≈0.7 V/Å, where D=0.39 debye for 3,3-dimethyl-1-butene. It is better to lower the temperature of dehydrogenation using a good catalyst substrate, as it can reduce the necessary magnitude of EF and assist in orienting the molecules. A more advanced method is to use strong laser pulses to excite targeted rotational modes of the molecules [29-31]. With an elliptically polarized field, one can not only align the molecular dipole, but also the orientation about the dipole [32], achieving the desired three-dimensional configuration of TAP.

With direct correspondence between the TAP and the relaxed structure, the approach can reveal the specific atomic connections needed to form a structure. The probability of forming selected metastable structure(s) can be further improved by deliberately inducing certain connection(s) between selective sites of the precursor units. According to FIG. 1, for penta-graphene, besides the existing sp³ C(4) of the precursor, C(1) becomes the other sp³ carbon by connecting to two C(3) and one C(E) in the neighbors. For R-graphene, O-graphene and graphene, some atoms of the neighboring units interact strongly due to their close distances with each other in the TAP, resulting in the breaking of an original bond of precursor during relaxations. Breaking the C(3)-C(4) single bond becomes more likely due to the presence of the strong C(1)-C(2) double bond. A complicated process is involved in forming graphene—not only C(E)-C(4) needs to break, but also each unit has to rotate to enable linkages from C(3) to C(E) and C(1).

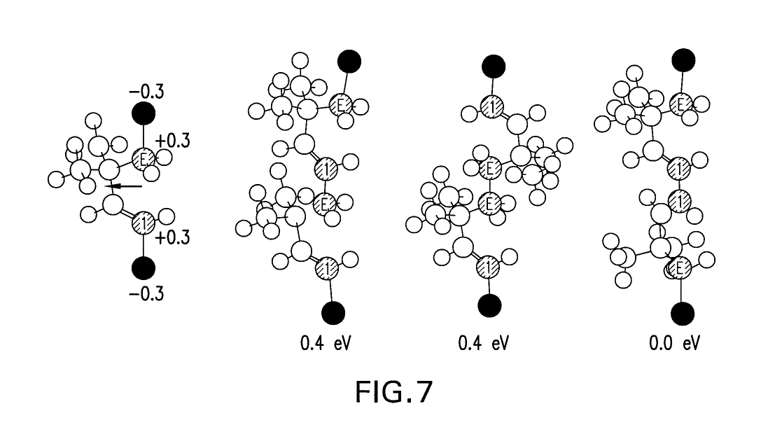

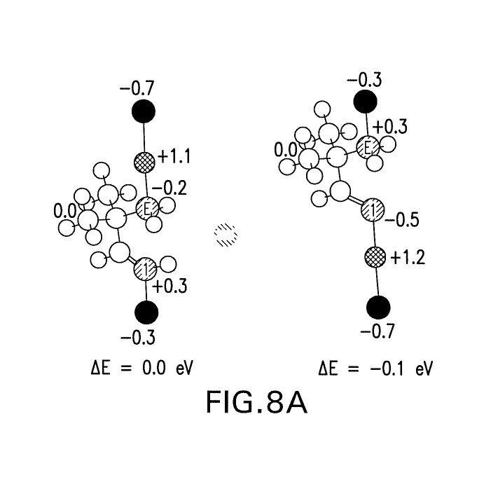

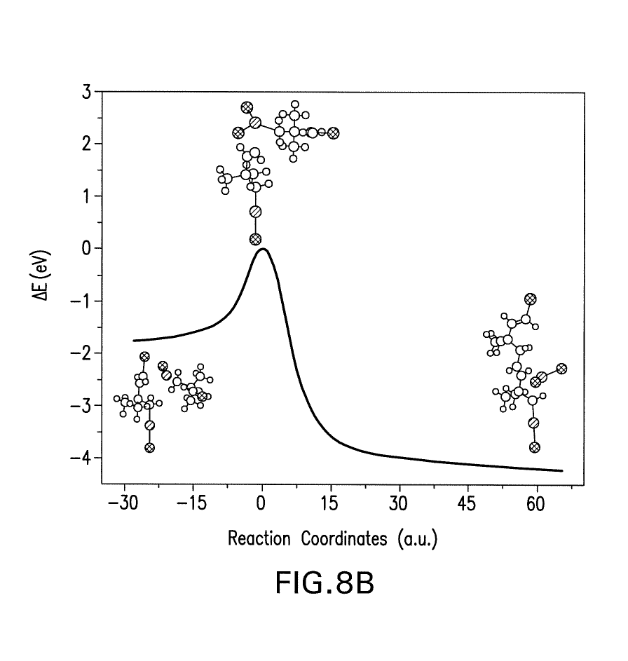

The connection between C(1) and C(E) is only needed for making penta-graphene and R-graphene. By introducing such connection, one can further enhance the probability of forming penta-graphene and R-graphene against the formation of graphene and other structures. One way is to introduce preferred connection between C(1) and C(E) through debromination (FIG. 7). Another way is to utilize the Grignard reaction. With C(1)-Br (or C(1)-Mg—Br) and C(E)-Mg—Br (or C(E)-Br) made in each molecule, C(1) and C(E) become highly positive and negative, respectively, so that C(1) will be drawn to C(E) in the reaction (see FIG. 8 [33-35]).

### REFERENCES

1. Falcao, E. H L. and Wudl, F. Carbon allotropes: beyond graphite and diamond. J. Chem. Technol. Biotechnol. 82, 524-531, 2007.
2. Custer, J. S.; Thompson, M. O.; Jacobson, D. C.; Poate, J. M.; Roorda, S.; Sinke, W. C.; Spaepen, F. Density of amorphous Si. Applied Physics Letters. 64, 437-439, 1994.
3. Zhou, X.-F.; Dong, X.; Oganov, A. R.; Zhu, Q.; Tian, Y. and H.-T. Wang, Semimetallic Two-Dimensional Boron Allotrope with Massless Dirac Fermions. Phys. Rev. Lett. 112, 085502, 2014.
4. Allain, A.; Kang, J.; Banerjee, K.; Kis, A., Electrical contacts to two-dimensional semiconductors. Nat. Mater. 14,1195-1205, 2015.
5. Liu, C.; Jiang, H.; Yao, Y. Low-energy effective Hamiltonian involving spin-orbit coupling in silicene and two-dimensional germanium and tin. Phys. Rev. B. 84, 195430, 2011.
6. Stevanovic, V. Sampling Polymorphs of Ionic Solids using Random Superlattices. Phys. Rev. Lett. 116, 075503, 2016.
7. Keskar, N. R. and Chelikowsky, J. R. Structural properties of nine silica polymorphs. Phys. Rev. B 46, 1, 1992.
8. Pannetier, J.; Bassas-Alsina, J.; Rodriguez-Carvajal, J. and Caignaert, V. Prediction of crystal structures from crystal chemistry rules by simulated annealing. Nature 346, 343, 1990.
9. Schoen, J. C. and Jansen, M. First Step Towards Planning of Syntheses in Solid-State Chemistry: Determination of Promising Structure Candidates by Global Optimization. Angew. Chem., Int. Ed. 35, 1286, 1996.
10. Wales, D. J. and Doye, J. P. K. Global Optimization by Basin-Hopping and the Lowest Energy Structures of Lennard-Jones Clusters Containing up to 110 Atoms. J. Chem. Phys. A 101, 5111, 1997.
11. Geodecker, S. Minima hopping: an efficient search method for the global minimum of the potential energy surface of complex molecular systems. J. Chem. Phys. 120, 9911, 2004.
12. Trimarchi, G. and Zunger, A. Global space-group optimization problem: Finding the stablest crystal structure without constraints. Phys. Rev. B 75, 104113, 2007.
13. Meredig, B. and Wolverton, C. A hybrid computational-experimental approach for automated crystal structure solution. Nat. Mater. 12, 123, 2013.
14. Tajima, N.; Tsuzuki, S.; Tanabe, K.; Aoki, K. and Hirano, T. First principles prediction of crystal structures of CO₂. Electron. J. Theor. Chem. 2, 139-148, 1997.
15. Fischer, C. C.; Tibbetts, K. J.; Morgan, D. and Ceder, G. Predicting crystal structure by merging data mining with quantum mechanics. Nat. Mater. 5, 641, 2006.
16. Dunk, P. W.; Kaiser, N. K.; Mulet-Gas, M.; Rodriguez-Fortea, A.; Poblet, J. M.; Shinohara, H.; Hendrickson, C. L.; Marshall, A. G. and Kroto, H. W. The smallest stable fullerene, M@C₂s (M=Ti, Zr, U): stabilization and growth from carbon vapor. J. Am. Chem. Soc. 134, 9380-9389 (2012).
17. Prinzbach, H. et al. C₂₀ carbon cluster: fullerene-boat-sheet generation, mass selection, photoelectron characterization. Chem. Eur. J. 12, 6268-6280 (2006).
18. Li, G.; Li, Y.; Liu, H.; Guo, Y.; Li, Y. and Zhu, D. Architecture of graphdiyne nanoscale films. chem. Commun. 46, 3256-3258, 2010.
19. Zhang, S.; Zhou, J.; Wang, Q.; Chen, X.; Kawazoe, Y. and Jena, P. Penta-graphene: a new carbon allotrope. PNAS 112, 2372-2377, 2015.
20. Su, C.; Jiang, H.; and Feng, J. Novel two-dimensional carbon allotrope with strong electronic anisotropy. Phys. Rev. B 87, 075453, 2013.
21. Schluetter, F.; Nishiuchi, T.; Enkelmann, V.; and Muellen, K. Octafunctionalized biphenylenes: molecular precursors for isomeric graphene nanostructures. Angew. Chem. Int. Ed. 53, 1538-1542, 2014.
22. Ewels, C. P.; Rocquefelte, X.; Kroto, H. W.; Rayson, M. J.; Briddon, P. R. and Heggie, M. I. Predicting experimentally stable allotropes: Instability of penta-graphene. PNAS 112, 15609-15612, 2015.
23. Pewdew, J. P., Burke, K., Ernzerhof, M. Generalized Gradient Approximation Made Simple. Phys. Rev. Lett. 77, 3865, 1996.
24. Kresse, G.; Furthmueller, J. Efficiency of ab-initio total energy calculations for metals and semiconductors using a plane-wave basis set. J. Comput. Mater. Sci. 6, 15-50, 1996.
25. Kresse, G.; Furthmueller, J. Efficient iterative schemes for ab initio total-energy calculations using a plane-wave basis set, Phys. Rev. B. 54, 11169-11186, 1996.
26. Grimme, S. Semiempirical GGA-type density functional constructed with a long-range dispersion correction. J. Comput. Chem. 27, 1787, 2006.
27. Fang, H.; Dove, M and Refson, K. Ag—Ag dispersive interaction and physical properties of Ag₃Co(CN)₆. Phys. Rev. B 90, 054302, 2014.
28. Liu, Y.; Wang, G.; Huang, Q.; Guo, L. and Chen, X. Structural and electronic properties of T graphene: a two-dimensional carbon allotrope with tetrarings. Phys. Rev. Lett. 108, 225505, 2012.
29. Friedrich, B. and Herschbach, D. Alignment and trapping of molecules in intense laser fields, Phys. Rev. Lett. 74, 4623, 1995.
30. Stapelfeldt, H. and Seideman, T. Colloquium: aligning molecules with strong laser pulses. Rev. Mod. Phys. 75, 543-557, 2003.
31. Chatterley, A. S.; Shepperson, B. and Stapelfeldt, H. Three-dimensional molecular alignment inside helium nanodroplets. Phys. Rev. Lett. 119, 073202, 2017.
32. Larsen, J. J.; Hald, K.; Bjerre, N.; Stapelfeldt, H. and Seideman, T. Three dimensional alignment of molecules using elliptically polarized laser fields, Phys. Rev. Lett. 85, 2470, 2000.
33. Frisch, M. J. et al. Gaussian 09, Revision A.02, Gaussian, Inc., Wallingford Conn., 2016.
34. Becke, A. Density-functional thermochemistry. III. The role of exact exchange. J. Chem. Phys. 98, 5648-5652, 1993.
35. Lee, C.; Yang, W.; Parr, R. G. Development of the colle-salvetti correlation-energy formula into a functional of the electron density. Phys. Rev. B. 37, 785-788, 1998.

### Example 2. Further Details of the Method Steps

1. Degrees of Freedom of the Precursor as a Rigid Body

A rigid body of the 3,3-dimethyl-1-butene has three rotational degrees of freedom and the three chosen rotational angles in the paper are considered as a good description. The six carbon atoms in the molecule have 3×6=18 degrees of freedom. There are 5 fixed bonds and 7 fixed angles between the carbon atoms. Therefore, the net degrees of freedom are 18−5−7=6, three of which are rotational degrees of freedom and the other three are translational. Only the rotational degrees of freedom are important herein, since only the orientation of the precursor molecules is considered. Thus, the three rotational degrees of freedom can be chosen as the two angles that can determine the orientation of a dipole vector and the third one as the rotational angle about the dipole vector.

### 2. Computational Methods to Generate FIG. 3 and FIG. 4 of Example 1

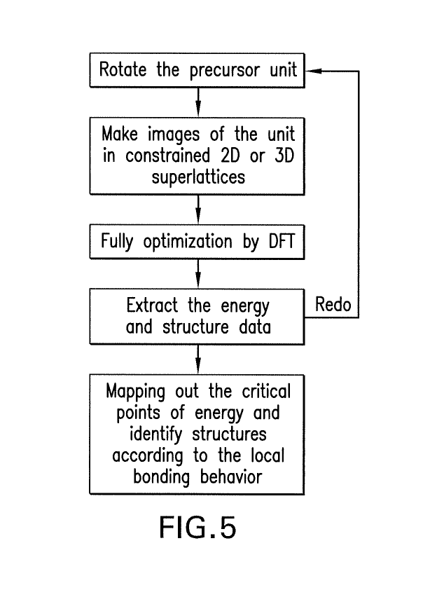

The work flow of computation is given in FIG. 5. 1000 calculations were carried out in the sampling process to obtain FIG. 3. Another 1000 calculations were carried out to produce the PES in FIG. 4. Calculations were conducted using Density Functional Theory (DFT) to optimize each TAP (with 54 atoms) to the closest critical point. Perdew-Burke-Ernzerhof (PBE) generalized gradient approximation (GGA) for exchange-correlation functional implemented in the VASP package was used. The projector augmented wave (PAW) pseudopotential method and a 2×2×2 Monkhorst-Pack k-point mesh were employed. The cutoff energy was 500 eV. The convergence of energy and force were set to 10⁻⁶ eV and 0.005 eV/Å, respectively. The van der Waals interaction (as implemented in the DFT+D3 method) was used during the optimization. Metastable states whose total energies are within 0.02 eV/atom were sorted together to generate the density of states in FIG. 3. To test the thermal stability of the areas of the targeted metastable structures on the energy map, ab initio molecular dynamics (AIMD) simulations up to 1000 K were conducted on the corresponding TAP with 1.0 fs time step until the structure reaches thermal equilibrium.

3. Specific Carbon Connections to Form Hybrid Structures with sp¹, sp² and sp³ Carbon.

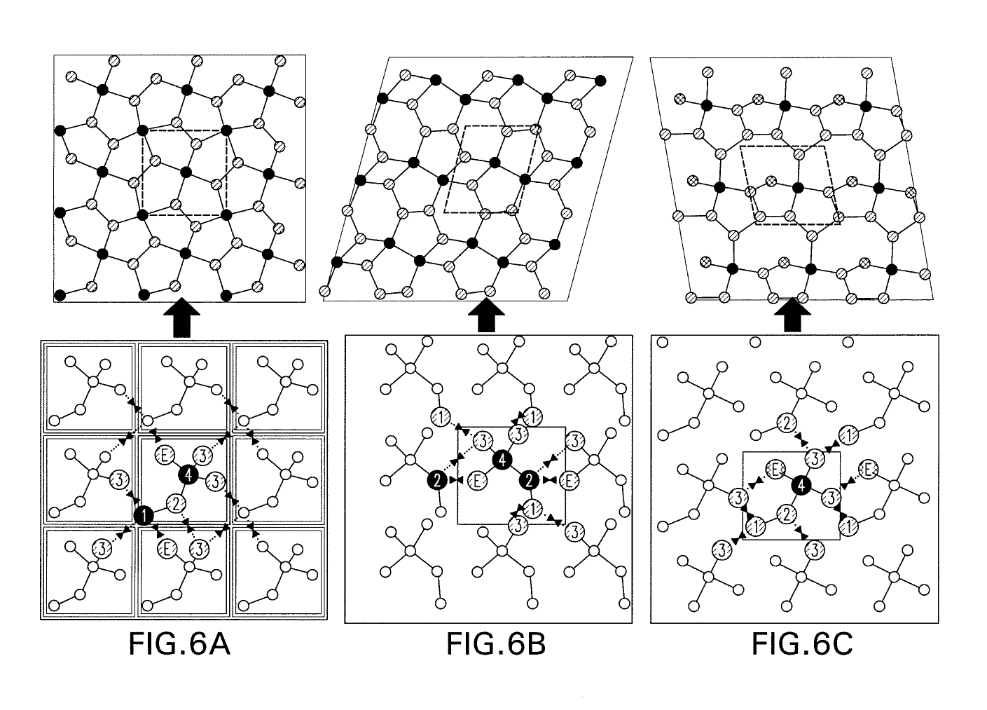

FIG. 6 shows the carbon-carbon connections needed to form penta-graphene, Q-graphene and E₂-graphene (as described e.g. in FIG. 3 and FIG. 4). No bond breaking is needed for these hybrid structures.

### 4. Forming C—C Connection Between Selective Sites of the Precursor Molecules Through the Dehalogenation and Grignard Reaction.

Cluster calculations are carried out using Gaussian 09. The hybrid density functional theory (DFT) with Becke three parameter Lee-Yang-Parr (B3LYP) prescription for the exchange-correlation energy and 6−31+G*basis sets are used. The optimized ground states correspond to the structures with the minimum energy and without any imaginary frequency. Atomic polar tensor-based (APT) charge analysis is used to obtain the charge states of atoms inside the cluster. The Grignard reaction pathway is calculated following intrinsic reaction coordinate (IRC). The results are shown in FIG. 7.

Due to the large concentration of charge on Br, the bromination of the molecule completely changes the direction of the original dipole moment which has a major component along C(1)-C(E). This compromises the ability to align the molecules along C(1)-C(E) by using, for example, an electric field. The desired C(1)-C(E) connection is not energetically favored compared to the C(1)-C(1) connection which is 0.4 eV lower in energy.

### Example 3. Synthesis of Penta-Graphene Using 3,3-dimethyl-1-butene as a Precursor

According to the steps described in Example 1, PES is constructed using TAP of 3,3-dimethyl-1-butene units, as shown in FIG. 4. On the PES, it is shown that penta-graphene exists in those striped areas with large γ values between the two high ridges, away from the depressions. The optimal condition to make penta-graphene is found to be around β=10°, where a basin of penta-graphene with a significant range [α=−10°<10°; γ=−10°˜10°] is established. The basin is fully enclosed by E₁ state representing aninterconversion barrier of 0.30 eV/atom. On its upper-left is a shallow depression corresponding to D-graphene comprised of three-, five- and ten-membered rings whose energy is 0.02 eV/atom higher. A structure of graphene with carbynes (linear chains of carbon) appears in a small-opening dip, which is isolated by wide surrounding peaks over 1.00 eV/atom. Thus, penta-graphene can be synthesized using the precursor 3,3-dimethyl-1-butene with TAP constructed in the orientations measured by the above these angles.

External fields such as electric field and laser pulse can help building the required TAP. Electric field can align the dipoles (axis D, FIG. 2D) of the precursor molecules to the chosen orientation. Laser pulse can fully control the orientation of the precursor molecules in the 3D space. The distance between the assembled precursor molecules can be controlled by vapor density in the experiment. According to FIG. 1A, inducing connection between certain carbon atoms of the neighboring 3,3-dimethyl-1-butene units can synthesize the penta-graphene. This can be done by using reactions, such as the dehalogenation and the Grinard reaction, as described in Example 2.

### Example 4. Steps to Select Precursors Using Computer Program

1. Import the crystal structure of the targeted metastable compounds (e.g. the periodic structure of penta-graphene by inputting its lattice parameters and atomic coordinates).
2. Read in the lattice vectors in three dimensions (length of the edge along each axis and the angle between the lettice vectors) and the number of atoms in each unit cell of the imported structure. Find the inequivalent atoms (in terms of the type and the local bonding symmetry, or the coordination number) in each unit cell (repetitive unit in the periodic structure). For example, in the case of penta-graphene, find that there are two sp³ (four coordinated) carbon atoms and four sp² (three coordinated) carbon atoms in each unit cell, where each sp³ carbon is coordinated by four sp² carbons.
3. Scan the database of molecular compounds for the selection of precursor using the following criteria: a) the total number of atoms in the molecule should match or be divisible by the number of atoms in the unit cell of the targeted metastable structure (e.g. the precursor 3,3-dimethyl-1-butene has 6 carbon atoms, which match the number of carbon in the unit cell of penta-graphene); b) the local bonding symmetry in the precursor molecule should match that of the unit cell of the targeted metastable structure (e.g. in the precursor 3,3-dimethyl-1-butene, the sp³ carbon is coordinated by four sp² carbon, which matches the case of the unit cell of penta-graphene).
4. For each precursor candidate found according to step 3, strip its extraneous atoms (e.g. hydrogen in the case of 3,3-dimethyl-1-butene) and match the skeleton of the precursor molecule to the atoms in the unit cell of the targeted metastable structure. This can be done using computational methods for registration of 3D shapes (Besl, J. P. and McKay, N. D. IEEE Transactions on Pattern Analysis and Machine Intelligence 14, 239, 1992), such as least-square fitting of two sets of 3D data sets. The precursor candidate results in the smallest fitting error will be chosen as the final candidate. For instance, in the case of penta-graphene, both 2,2-dimethylbutane and 3,3-dimethyl-1-butene are potential candidates from step 3. However, after fitting the precursor skeleton to the atomic arrangement in the unit cell of penta-graphene, it is found that one dihedral angle formed by four carbon atoms in 2,2-dimethylbutane is too large, resulting in a large fitting error. Therefore, 3,3-dimethyl-1-butene rather than 2,2-dimethylbutane is selected as the final candidate for the synthesis of penta-graphene.
5. Repeating steps 3-4 for different molecular database until at least one precursor for the targeted metastable structure(s) is selected. Building TAP using the selected precursor molecules as exemplified in EXAMPLE 1, subject to DFT relaxation to the nearest critical point of energy.
6. Read in the DFT calculated energy for different TAP. Draw the PES according to the three angles around the chosen rotational axes (e.g. axes x and z as well as the dipole axis of 3,3-dimethyl-1-butene in the case of penta-graphene) that used to define each TAP.
7. Computing the frequency of occurrence of a metastable structure by counting how many times its corresponding energy appear in the constructed PES in step 6. Find out all the TAP that will end up with the targeted metastable structure(s).
8. Analyze the bonding characteristics between each precursor molecule and its nearest neighbors and find out the connection(s) needed between specific atomic nodes in order to form the targeted metastable structure(s).
9. Output the constructed PES, frequency of occurrence of each metastable structure, and the bonding characteristics from step 6-8 to guide the experiment.

### Example 5. List of Examples of 2D and 3D Metastable Crystalline Structures that could be Made from Precursors Selected by the Method

2D structures included but not limited to (written in the format “name, type of rings in the structure, reference” in each case):

1. α-graphyne, 18, [1];
2. graphyne, 6+12, [2];
3. 8-graphyne, 6+14, [3];
4. 3-graphyne, 12+18, [1];

### 5. T-graphene, 4+8, [4];

### 6. Pentaheptite, 5+7, [5];

### 7. OPZ-L/Z, 5+8, [5];

### 8. C31, 3+9, [5];

### 9. C41, 4+7, [6];

### 10. C63, 3+6+8, [6];

### 11. H-net, 4+6+8, [7];

12. net-W, 4+6+8, [8];

### 13. S-graphene, 4+6+10, [9];

14. BPC graphenylene, 4+6+12, [10];
15. pza-C10, 5+6+7, [11];

### 16. Dimerite, 5+6+7, [12];

17. Haeckelite structures, 5+6+7, [13];

### 18. HOPG, 5+6+8, [14];

### 19. Octite, 5+6+8, [15];

### 20. C65, 5+6+9, [6].

3D structures included but not limited to (written in the format “name, possible precursor, reference” in each case)

### 1. T-carbon [16];

### 2. L-carbon [17];

### 3. Y-carbon [18];

4. cubane-based carbon [19];
5. T6-carbon, acenes or polyacenes, [20];
6. T12-carbon, alkane or alkene molecules, [21];
7. T14-carbon, acenes or polyacenes, [20].

### REFERENCES

1. Malko D, Neiss C, Viñes F, Görling A (2012) Competition for graphene: graphynes with direction-dependent Dirac cones. Phys Rev Lett 108(8):086804.
2. Narita N, Nagai S, Suzuki S, Nakao K (1998) Optimized geometries and electronic structures of graphyne and its family. Phys Rev B 58(16):11009-11014.
3. Zhao M, Dong W, Wang A (2013) Two-dimensional carbon topological insulators superior to graphene. Sci Rep 3:3532.
4. Liu Y, Wang G, Huang Q, Guo L, Chen X (2012) Structural and electronic properties of T-Graphene: a two-dimensional carbon allotrope with tetrarings. Phys Rev Lett 108(22):225505.
5. Merz K M, Hoffmann R, Balaban A T (1987) 3,4-connected carbon nets: through-space and through-bond interactions in the solid state. J Am Chem Soc 109(22):6742-6751.
6. Lu H, Li S-D (2013) Two-dimensional carbon allotropes from graphene to graphyne. J Mater Chem C 1(23):3677-3680.
7. Hu M, et al. (2014) Theoretical two-atom thick semiconducting carbon sheet. Phys Chem Chem Phys.
8. Wang X-Q, Li H-D, Wang J-T (2013) Prediction of a new two-dimensional metallic carbon allotrope. Phys Chem Chem Phys 15(6):2024-2030.
9. Xu L C, et al. (2014) Two dimensional Dirac carbon allotropes from graphene. Nanoscale 6(2):1113-1118.
10. Brunetto G, et al. (2012) Nonzero gap two-dimensional carbon allotrope from porous graphene. J Phys Chem C 116(23):12810-12813.
11. Luo X, et al. (2012) Two-dimensional superlattice: modulation of band gaps in graphene-based monolayer carbon superlattices. J Phys Chem Lett 3(22):3373-3378.
12. Lusk M T, Carr L D (2009) Creation of graphene allotropes using patterned defects. Carbon 47(9):2226-2232.
13. Terrones H, et al. (2000) New metallic allotropes of planar and tubular carbon. Phys Rev Lett 84(8):1716-1719.
14. Mandal B, Sarkar S, Pramanik A, Sarkar P (2013) Theoretical prediction of a new two-dimensional carbon allotrope and NDR behaviour of its one-dimensional derivatives. Phys Chem Chem Phys 15(48):21001-21006.
15. Appelhans D J, Lin Z, Lusk M T (2010) Two-dimensional carbon semiconductor: density functional theory calculations. Phys Rev B 82(7):073410.
16. Sheng X-L, Yan Q-B, Ye F, Zheng Q-R, Su G (2011) T-carbon: A novel carbon allotrope. Phys Rev Lett 106(15):155703.
17. Yang L, He H Y, Pan B C (2013) Theoretical prediction of new carbon allotropes. J Chem Phys 138(2):024502-024506.
18. Jo J Y, Kim B G (2012) Carbon allotropes with triple bond predicted by first-principle calculation: Triple bond modified diamond and T-carbon. Phys Rev B 86(7):075151.
19. Srinivasu K, Ghosh S K (2012) Electronic structure, optical properties, and hydrogen adsorption characteristics of supercubane-based three-dimensional porous carbon. J Phys Chem C 116(47):25015-25021.
20. Zhang S, Wang Q, Chen X, Jena P (2013) Stable three-dimensional metallic carbon with interlocking hexagons. PNAS 110(47):18809-18813.
21. Zhao Z et al. (2012) Tetragonal allotrope of group 14 elements. J Am Chem Soc 134(30):12362-12365.

While the invention has been described in terms of its several exemplary embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method of synthesizing a metastable crystalline material from a precursor comprising
I) selecting the precursor by:
i) identifying potential precursors, wherein the potential precursors are identified by
determining the number of atoms in a building block of the metastable crystalline material;
determining the types of bonds between the atoms in the building block;
selecting, from a molecular database, potential precursors having
a) the same type of atoms as the builiding block,
b) the same number of atoms as the builiding block, and
c) at least one bond of a type that is the same as at least one bond in the building block;
aligning the potential precursors;
selecting, as candidate precursors, potential precursors in which bonding between atoms of aligned neighboring potential precursors can occur;
ii) for each selected candidate precursor, generating a set of different topologically aligned precursors (TAP);
iii) geometrically and ionically relaxing each TAP to a closest critical point of the potential energy surfaces (PES);
iv) calculating the frequency of occurrence of the metastable crystalline material within relaxed TAP;
v) selecting at least one candidate precursor to be used as a precursor to synthesize the metastable crystalline material, wherein a frequency of occurrence of the at least one metastable crystalline material in the relaxed TAP of that candidate precursor is at least 2 times a frequency of occurrence of energetically similar metastable crystalline structures, and/or the ground-state of the metastable crystalline structure; and
II) reacting the precursor to form the metastable crystalline material.

2. The method of claim 1, wherein the metastable crystalline material is a two dimensional (2D) or three dimensional (3D) metastable crystalline material, or an allotrope thereof.

3. The method of claim 1, wherein the metastable crystalline material comprises one or more of carbon, boron, nitrogen, phosphorus, silicon or a metal.

4. The method of claim 1, wherein the metastable crystalline material is a 2D carbon allotrope.

5. The method of claim 4, wherein the 2D carbon allotrope is penta-graphene, O-graphene or R-graphene.

6. The method of claim 1, wherein the step of geometrically and ionically relaxing is performed using density functional theory (DFT).

7. The method of claim 1, wherein the step of geometrically and ionically relaxing is performed while suppressing accessibility of the ground state and other isomers.

8. The method of claim 1, wherein each TAP is formed by contraining, within a superlattice, multiple copies of one candidate precursor.

9. The method of claim 8, wherein each copy of the candidate precursor within the superlattice has the same fixed orientational configuration.

10. The method of claim 1, wherein the frequency of occurrence is at least twice the frequency of occurrence of energetically nearest neighbor structures and/or the ground-state structure.

11. The method of claim 1, wherein the frequency of occurrence is at least one order of magnitude higher than the frequency of occurrence of energetically nearest neighbor structures and/or the ground-state structure.

12. The method of claim 1, wherein the step of selecting comprises selecting potential precursors having at least one of:
the same type of atoms as the building block of the metastable crystalline material,
the same number of atoms as the building block of the metastable crystalline material,
the same atomic orbitals as the building block of the metastable crystalline material, and
the same size as the building block of the metastable crystalline material.

13. The method of claim 1, wherein the metastable crystalline material is pentagraphene and the precursor that is selected is 3,3-dimethyl-1-butene.

14. A method of selecting a precursor for synthesis of a metastable crystalline structure, comprising
i) identifying potential precursors, wherein the potential precursors are identified by
determining the number of atoms in a building block of the metastable crystalline material;
determining the types of bonds between the atoms in the building block;
selecting, from a molecular database, potential precursors having
a) the same type of atoms as the builiding block,
b) the same number of atoms as the builiding block, and
c) at least one bond of a type that is the same as at least one bond in the building block;
aligning the potential precursors;
selecting, as candidate precursors, potential precursors in which bonding between atoms of aligned neighboring potential precursors can occur;
ii) for each selected candidate precursor, generating a set of different topologically aligned precursors (TAP);
iii) geometrically and ionically relaxing each TAP to a closest critical point of the potential energy surfaces (PES);
iv) calculating the frequency of occurrence of the metastable crystalline material within relaxed TAP; and
v) selecting at least one candidate precursor to be used as a precursor to synthesize the metastable crystalline material, wherein a frequency of occurrence of the at least one metastable crystalline material in the relaxed TAP of that candidate precursor is at least 2 times a frequency of occurrence of energetically similar metastable crystalline structures, and/or the ground-state of the metastable crystalline structure.

15. A compound having a metastable crystalline structure, wherein the compound is selected from the group consisting of penta-graphene, O-graphene and R-graphene.
